(19) United States
(12) Patent Application Publication
(10) Pub. No.: US 2023/0248634 A1
(43) Pub. Date: Aug. 10, 2023

(54) UV-BLOCKING COSMETIC COMPOSITION HAVING INCREASED UV-BLOCKING EFFICIENCY DUE TO HEAT AND UV LIGHT

(71) Applicant: LG HOUSEHOLD & HEALTH CARE LTD., Seoul (KR)

(72) Inventors: Hyeong-Jin CHO, Seoul (KR); Min-Sung CHOI, Seoul (KR); Seung-Jin SONG, Seoul (KR)

(73) Assignee: LG HOUSEHOLD & HEALTH CARE LTD., Seoul (KR)

(21) Appl. No.: 18/000,821

(22) PCT Filed: May 24, 2021
(86) PCT No.: PCT/KR2021/006456

§ 371 Date: Dec. 5, 2022

(30) Foreign Application Priority Data
Jun. 3, 2020 (KR) 10-2020-0067122
Dec. 2, 2020 (KR) 10-2020-0166725

(51) Int. Cl. A61K 8/81; A61K 8/34; A61Q 17/04

(52) U.S. Cl. CPC A61K 8/8152; A61K 8/345; A61Q 17/04; A61K 2800/48

(57) ABSTRACT

The present disclosure relates to a composition for increasing ultraviolet blocking efficiency of an ultraviolet blocking agent by heat. In general, the UV blocking efficiency of the UV blocking agent is reduced by heat, but the present disclosure shows an unexpected effect in that the UV blocking efficiency of the UV blocking agent is increased by heat.

### TECHNICAL FIELD

The present application claims the priority based on Korean Patent Application No. 10-2020-0067122 filed on Jun. 3, 2020 and Korean Patent Application No. 10-2020-0166725 filed on Dec. 2, 2020, and the entire contents disclosed in the description and drawings of the corresponding applications are incorporated in the present application.

The present disclosure relates to a method, composition, use or application for increasing ultraviolet blocking efficiency of a UV blocking agent by heat.

### BACKGROUND ART

Ultraviolet rays irradiated from sunlight act as a major cause of erythema, edema, freckles or skin cancer on skin. Recently, many studies on various skin diseases caused by UV rays are being actively conducted. In general, UV rays are classified according to their wavelength, which are that 200-280 nm wavelength is UV-C, 280-320 nm wavelength is UV-B, or 320-400 nm wavelength is UV-A. UV-C passes through the ozone layer and is lost without reaching the earth surface, and UV-B penetrates to the epidermis of skin and causes erythema, freckles or edema or the like. UV-A is known to penetrate into the dermis of skin and promote skin cancer and wrinkles, and melanin formation, and cause skin irritation. Numerous epidemiological studies have demonstrated a strong relationship between sun exposure and human skin cancer.

As a result of the above-mentioned risk related to sun exposure, public interest in UV blocking products has increased, and as a result, various kinds of UV blocking products with SPF (sun protection factor) have been launched. The UV blocking products comprise inorganic UV blocking agents such as titanium dioxide or zinc oxide or the like, or organic UV blocking agents such as ethylhexylmethoxycinnamate, ethylhexylsalicylate, octocrylene, butylmethoxydibenzoylmethane, bis-ethylhexyloxyphenolmethoxyphenyltriazine, or diethylaminohydroxybenzoylhexylbenzoate, or the like, in order to have a high SPF value.

On the other hand, it has been raised that the UV blocking agent applied to skin has a problem in that it cannot fulfill its role due to damage to the blocking film due to moisture and deterioration of photostability due to ultraviolet rays. In particular, ethylhexylmethoxycinnamate, isoamyl p-methoxycinnamate, or cinoxate which is a UV blocking agent having a methoxycinnamate structure, is known to have a problem in that the efficiency of absorbing light in the ultraviolet region decreases, as the trans structure is changed to a cis structure by ultraviolet rays.

Recently, technology to solve this problem of decreasing the UV blocking efficiency has been developed, but it is mainly related to solving the problem of decreasing the UV blocking efficiency, and studies to improve the deterioration of the UV blocking effect caused by heat are insufficient.

As the summer temperature increases every year, UV blocking agents corresponding thereto are required. When the UV blocking efficiency is lowered by heat, UV rays cannot be effectively blocked, and therefore, skin aging or various kinds of skin diseases may be caused. In addition, as skin exposure to sunlight increases due to the increase in outdoor activities, it may cause inconvenience of having to reapply UV blocking cosmetics several times. In order to solve this problem, there is a need to develop a method for preventing a decrease of the effect of the UV blocking agent itself by heat or a method for further enhancing the effect in spite of using the same amount.

Numerous documents are referenced throughout the present description and citations thereof are indicated. The disclosures of the cited documents are incorporated in the present description in their entirety to more clearly set forth the level of the art to which the present disclosure pertains and the content of the present disclosure.

### DISCLOSURE

### Technical Problem

As a result of intensive research efforts to solve the problems of the prior art, the inventors of the present disclosure have found that the ultraviolet blocking efficiency of the UV blocking agent is rather increased when applying heat in case of using a self-emulsifying polymer and a polyol having an IOB value of 5.0 or less, thereby completing the present disclosure.

Accordingly, objects of the present disclosure are to provide the following embodiments.

Embodiment 1. A method of increasing UV blocking efficiency of a UV blocking agent by heat, using (a) a self-emulsifying polymer and (a) a polyol having an IOB value of 5.0 or less.

Embodiment 2. The method according to Embodiment 1, wherein the method comprises preparing a water phase comprising the self-emulsifying polymer and the polyol; preparing an oil phase comprising the UV blocking agent; preparing a mixed composition by mixing the water phase and oil phase; and applying heat to the mixed composition.

Embodiment 3. A composition for use in increasing UV blocking efficiency of a UV blocking agent by heat, comprising a combination of (a) a self-emulsifying polymer and (a) a polyol having an IOB value of 5.0 or less as an active ingredient; a use of a combination of (a) a self-emulsifying polymer and (a) a polyol having an IOB value of 5.0 or less for increasing UV blocking efficiency of a UV blocking agent by heat; or a use of a combination of (a) a self-emulsifying polymer and (a) a polyol having an IOB value of 5.0 or less for preparing a cosmetic with increased blocking efficiency by heat.

Embodiment 4. The method, composition, or use according to any one of the preceding embodiments, wherein the active ingredient which plays a role of increasing UV blocking efficiency of a UV blocking agent by heat consists of a combination of (a) a self-emulsifying polymer and (a) a polyol having an IOB value of 5.0 or less.

Embodiment 5. The method, composition, or use according to any one of the preceding embodiments, wherein the self-emulsifying polymer is at least one selected from the group consisting of sodium acrylate/beheneth-25 methacrylate cross-polymer, sodium acrylate/sodium acryloyldimethyltaurate copolymer, sodium acylate/acryloyldimetatrate/dimethylacrylamide cross-polymer, sodium polyacrylate, acrylate/C10-30 alkylacrylate cross-polymer, acrylate/beheneth-25 methacrylate copolymer, acrylate copolymer, ammonium acryloyldimethyltaurate/VP copolymer, polyacrylate-13, polyacrylate cross-polymer-6, polyacrylamide, PEG-240/HDI copolymer bis-decyltetradeceth-20 ether, and hydroxyethylacrylate/sodium acryloyldimethyltaurate copolymer.

Embodiment 6. The method, composition, or use according to any one of the preceding embodiments, wherein the polyol having an IOB value of 5.0 or less is at least one selected from the group consisting of glycerin, sorbitol, xylitol, glucose, trehalose, diglycerin, propandiol, propylene glycol, polyglycerin-3, methylpropandiol, butylene glycol, pentylene glycol, PEG-6, PEG-8, glycereth-26, dipropylene glycol, 1,2-hexanediol and caprylyl glycol.

Embodiment 7. The method, composition, or use according to any one of the preceding embodiments, wherein the UV blocking agent is at least one selected from the group consisting of aminobenzoic acid-based compounds, benzophenone-based compounds, cinnamate-based compounds, salicylate-based compounds, inorganic metal oxides-based compounds, butyl methoxydibenzoylmethane-based compounds, terephthalylidene dicamphor sulfonic acid ecamsule, phenylbenzimidazole sulfonic acid, bemotrizinol and bisoctrizole.

Embodiment 8. The method, composition, or use according to any one of the preceding embodiments, wherein the self-emulsifying polymer is an aqueous thickener.

Embodiment 9. The method, composition, or use according to any one of the preceding embodiments, wherein the composition comprises a water phase and an oil phase, and the (a) self-emulsifying polymer, and (b) polyol having an IOB value of 5.0 or less are comprised in the water phase, and the UV blocking agent is comprised in the oil phase.

Embodiment 10. The method, composition, or use according to any one of the preceding embodiments, wherein the composition has a more increased synergistic effect of the UV blocking efficiency of the UV blocking agent by heat, when UV rays are irradiated.

Embodiment 11. A cosmetic comprising the composition according to any one of the preceding embodiments.

Other objects and advantages of the present disclosure will become more apparent from the following detailed description of the invention, claims and drawings.

### Technical Solution

One aspect of the present disclosure is to provide a method for increasing UV blocking efficiency of a UV blocking agent by heat, using (a) a self-emulsifying polymer and (a) a polyol having an IOB value of 5.0 or less.

The method may comprise preparing a water phase comprising the self-emulsifying polymer and the polyol; preparing an oil phase comprising the UV blocking agent; preparing a mixed composition by mixing the water phase and oil phase; and applying heat to the mixed composition.

Another aspect of the present disclosure is to provide a composition for increasing UV blocking efficiency of a UV blocking agent by heat, comprising a combination of (a) a self-emulsifying polymer and (a) a polyol having an IOB value of 5.0 or less as an active ingredient.

Self-Emulsifying Polymer

The self-emulsifying polymer (SEP) of the present disclosure means a polymer which simultaneously has hydrophilic and hydrophobic groups in the chain and has an emulsifying ability and a thickening ability at the same time, and when an emulsion composition is prepared using the self-emulsifying polymer, a stable composition can be prepared without any additional emulsifier, and the UV blocking agent film can be spread when heat is applied after application, and consequently, the UV blocking region is widely uniformed by heat, so it is possible to realize the effect of increasing the effect. On the other hand, in case of general emulsification, even when heat is applied, the emulsifier located at the interface interferes with the movement of the UV blocking agent, so there is no change in the blocking effect before and after heating. Compared to this general emulsification, the principle of increasing the UV blocking effect by the polymeric emulsification of the present disclosure is briefly shown in FIG. 1.

The example of the self-emulsifying polymer includes sodium acrylate/beheneth-25methacrylatecross-polymer, sodium acrylate/sodium acryloyldimethyltaurate copolymer, sodium acrylate/acryloyldimetatrate/dimethylacrylamidecross-polymer, sodium polyacrylate, acrylate/C10-30 alkylacrylatecross-polymer, acrylate/beheneth-25 methacrylate copolymer, acrylate copolymer, ammonium acryloyldimethyltaurate/beheneth-25methacrylatecross-polymer, ammonium acryloyldimethyltaurate/VP copolymer, poly acrylate-13, poly acrylatecross-polymer-6, poly acrylamide, PEG-240/HDI copolymerbis-decyltetradeceth-20ether, hydroxyethylacrylate/sodium acryloyldimethyltaurate copolymer, and the like, but not limited thereto.

The self-emulsifying polymer may be comprised in an amount of 0.01% by weight to 10% by weight in the composition of the present disclosure.

Polyol having IOB value of 5.0 or less

The IOB (inorganic-organic balance) value is a numerical value indicating hydrophilicity of an organic compound, and the larger the value, the higher the hydrophilicity. For the IOB value, the organic value (OV) and inorganic value (IV) are calculated, based on the organic and inorganic table described in ┌Organic Conceptual Diagram—Basics and Applications┘ (written by Koda Yoshio (1984) published by Sankyo) and the like, and thereby, it is possible to calculate IOB value as IOB value=(inorganic value/organic value).

In case of the polyol, even after the moisture evaporates after application, it remains in pores where the water phase is located, and when heat is applied thereto, due to the compatibility of the polyol and the oil phase comprising UV blocking agent, the UV blocking agent can be spread in the pores, and consequently, an effect of increasing the UV blocking efficiency is caused with the effect of making the UV blocking region widely uniform. In order to facilitate this diffusion, the oil phase of the composition according to the present disclosure preferably has a viscosity of 1,000 cps or less, more preferably, 500 cps or less (measurement condition: Brookfield viscometer, 40° C., LVF spindle #1, 30 rpm, 1 min). In addition, the viscosity of the oil components among components mixed in the oil phase is preferably 1,000 cps or less, more preferably, 500 cps or less (measurement condition: Brookfield viscometer, 40° C., LVF spindle #1, 30 rpm, 1 min).

The IOB value of ethanol, and the IOB value of the generally widely used polyols are summarized in Table 1 below.

TABLE 1

| Raw material name | OV | IV | IOB |
|---|---|---|---|
| Glycerin | 60 | 300 | 5.00 |
| Sorbitol | 120 | 600 | 5.00 |
| Xylitol | 100 | 500 | 5.00 |
| Glucose | 120 | 530 | 4.42 |
| Trehalose | 240 | 880 | 3.67 |
| Diglycerin | 120 | 420 | 3.50 |
| Propanediol | 60 | 200 | 3.33 |
| Polyglycerin-3 | 180 | 540 | 3.00 |
| Methylpropanediol | 70 | 200 | 2.86 |
| Ethanol | 40 | 100 | 2.50 |
| Butylene glycol | 80 | 200 | 2.50 |
| PEG-6 | 240 | 575 | 2.40 |
| PEG-8 | 320 | 725 | 2.27 |
| Glycereth-26 | 180 | 360 | 2.00 |
| Dipropylene glycol | 120 | 220 | 1.83 |
| 1,2-hexanediol | 120 | 200 | 1.67 |

The polyol having an IOB value of 5.0 or less used in the present disclosure may be for example, at least one selected from the group consisting of glycerin, sorbitol, xylitol, glucose, trehalose, diglycerin, propandiol, propylene glycol, polyglycerin-3, methylpropandiol, butylene glycol, pentylene glycol, PEG-6, PEG-8, glycereth-26, dipropylene glycol, 1,2-hexanediol and caprylyl glycol, but not limited thereto. The polyol having an IOB value of 5.0 or less may be comprised in an amount of 0.1% by weight to 50% by weight in the composition of the present disclosure.

UV Blocking Agent

In general, as the UV blocking agent, both organic UV blocking agents having a UV absorbing effect and inorganic UV blocking agents having a scattering effect can be used regardless of the types.

The UV blocking agent may be at least one selected from the group consisting of aminobenzoic acid-based compounds, benzophenone-based compounds, cinnamate-based compounds, salicylate-based compounds, inorganic metal oxides-based compounds, butyl methoxydibenzoylmethane-based compounds, terephthalylidene dicamphor sulfonic acid ecamsule, phenylbenzimidazole sulfonic acid, bemotrizinol and bisoctrizole, but not limited thereto.

Examples of the aminobenzoic acid-based compound includes PABA, glyceryl PABA, Padimate O, Roxadimate, and the like, and examples of the benzophenone-based compound includes dioxybenzone, oxybenzone, sulisonbenzone and the like, and examples of the cinnamate-based compound includes octocrylene, octyl methoxycinnamate (octinoxate), ethoxyethyl p-methoxycinnamate (cinoxate), and the like, and examples of the salicylate-based compound includes homomenthyl salicylate (homosalate), ethylhexyl salicylate (octyl salicylate/octisalate), trolamine salicylate, and the like, and the inorganic metal oxides includes titanium dioxide, or zinc oxide, or the like, but not limited thereto.

The UV blocking agent may be comprised in an amount of 0.5% by weight to 30% by weight in the composition of the present disclosure.

In one embodiment of the present disclosure, the active ingredient which plays a role of increasing UV blocking efficiency of a UV blocking agent by heat may consist of (a) a self-emulsifying polymer and (b) a polyol having an IOB value of 5.0 or less.

In another embodiment of the present disclosure, the composition for increasing UV blocking efficiency of a UV blocking agent by heat of the present disclosure may comprise a water phase and an oil phase, and herein, the self-emulsifying polymer is an aqueous thickener, and is comprised in the water phase with the polyol having an IOB value of 5.0 or less, and the UV blocking agent may be comprised in the oil phase.

In another embodiment, the composition for increasing UV blocking efficiency of a UV blocking agent by heat of the present disclosure may not substantially comprise an oil phase thickener such as dextrin palmitate, disteadimonium hectorite, stearoyl inulin, polyamide-8, glycerylbehenate/eicosadioate and the like. Herein, the meaning of ‘substantially not comprising an oil phase thickener’ means that the composition does not comprise an oil phase thickener, or even if an oil phase thickener is comprised, it can be comprised in an amount or less that makes the viscosity of the oil phase less than or equal to 1,000 cps, more preferably, less than or equal to 500 cps (measurement condition: Brookfield viscometer, 40° C., LVF spindle #1, 30 rpm, 1 min). This is to facilitate diffusion of the composition components into the pores generated when the water phase volatilizes after the composition according to the present disclosure is applied to the skin.

In another embodiment, the composition for increasing UV blocking efficiency of a UV blocking agent by heat of the present disclosure, may not comprise any other emulsifier component other than the self-emulsifying polymer of the present disclosure.

Furthermore, the composition of the present disclosure may further comprise a moisturizer, a thickener, a surfactant, an oil phase base, a preservative, an antioxidant, an alcohol, a flavoring, a pH adjusting agent or a natural extract or the like, but not limited thereto. According to the present disclosure, when this additional component is comprised, the viscosity of the oil phase is preferably 1,000 cps or less, more preferably, 500 cps or less (measurement condition: Brookfield viscometer, 40° C., LVF spindle #1, 30 rpm, 1 min).

In general, the UV blocking efficiency of the UV blocking agent is reduced by heat, but when the active ingredient of the present disclosure, the combination of the (a) self-emulsifying polymer and (b) polyol having an IOB value of 5.0 or less is used, the UV blocking efficiency of the UV blocking agent is rather increased by heat.

The composition for increasing the UV blocking efficiency of the UV blocking agent by heat of the present disclosure has a significantly increased SPF value after applying heat of 40° C. for 15 minutes, and specifically, compared to that before applying heat, it may increase by at least 1%, at least 5%, at least 10%, at least 15%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, or at least 390%, and it may increase even as much as 400%.

Moreover, the composition of the present disclosure shows a synergistic effect in relation to the increase in the UV blocking efficiency when UV irradiation is added in addition to heat.

Other aspect of the present disclosure is to provide a cosmetic comprising the afore-mentioned composition for increasing UV blocking efficiency of the UV blocking agent by heat.

The cosmetic may be prepared in any formulation conventionally prepared in the art, and for example, it may be formulated as a solution, suspension, emulsion, paste, gel, cream, lotion, soap, surfactant-containing cleansing, oil and the like, but not limited thereto.

More specifically, the cosmetic may be prepared as a formulation such as mist, serum, nourishing cosmetic water, soft cosmetic water, soft water, emulsion, astringent, toner, skin, lotion, cream, foam, makeup base, essence, liquid detergent, bathing agent, sunscreen cream, sun oil, sunscreen emulsion, sunblock product, makeup product, makeup base, foundation, makeup pact, BB cream, CC cream, makeup powder, and the like, but not limited thereto.

All components described in the present disclosure, preferably, do not exceed the maximum use value stipulated in relative laws and regulations (for example, regulations on cosmetic safety standards, and the like (Korea), cosmetic safety technical standards (China)) and the like of Korea, China, U.S., Europe, Japan and the like. In other words, preferably, the cosmetic, cosmetic product or composition of the present disclosure according to the present disclosure comprises the components according to the present disclosure within the content limit allowed by relative laws and regulations of various countries.

### Advantageous Effects

In general, the ultraviolet blocking efficiency of the UV blocking agent is reduced by heat, but the present disclosure provides a technology which rather increases the UV blocking efficiency of the UV blocking agent by heat.

### BRIEF DESCRIPTION OF THE DRAWINGS

FIG. 1 is a schematic diagram showing the principle of the synergistic UV blocking effect by the polymeric emulsification of the present disclosure compared to general emulsification. In case of general emulsification, even when heat is applied, the emulsifier located at the interface interferes with the movement of the UV blocking agent, so there is no change in the blocking effect before and after heating, but in case of polymeric emulsification, when the mobility of the UV blocking agent is increased due to temperature rise, the fluidity of the interface is large, so it does not interfere with the movement of the UV blocking agent and therefore the UV blocking area after heating expands and the blocking effect increases.

FIG. 2 is a schematic diagram showing the function of the polyol having an IOB value of 5.0 or less of the present disclosure. When there is no polyol in the water phase, expansion of the UV blocking agent cannot occur when heated because the water phase is completely dried, but when polyol is included in the water phase, the polyol remains in the water phase even after drying and expansion of the UV blocking agent into the water phase during heating is possible due to compatibility between the polyol and UV blocking agent, and therefore, an increase in the blocking effect occurs.

FIG. 3 is a graph showing the result of evaluating the effects on the UV blocking effect of polymeric emulsification and general emulsification.

FIG. 4 shows the result of evaluating the compatibility according to the polyol type.

FIG. 5 shows the result of calculating the SPF change rate after preparing the UV blocking composition and heating to determine the effect of whether or not comprising the emulsifier.

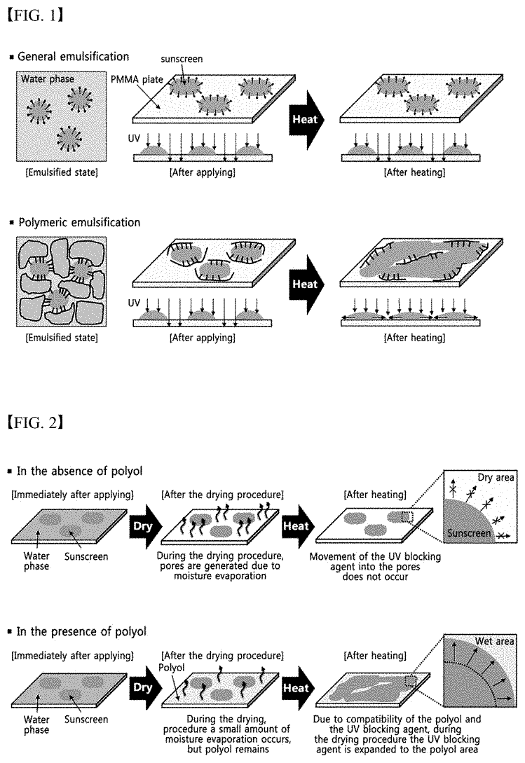

### MODE FOR INVENTION

Hereinafter, the present disclosure will be described in more detail by examples. These examples are intended only to illustrate the present disclosure more specifically, and it will be obvious to those skilled in the art that the scope of the present disclosure is not limited by these examples.

### Example

1. Comparison of Polymeric Emulsification and General Emulsification

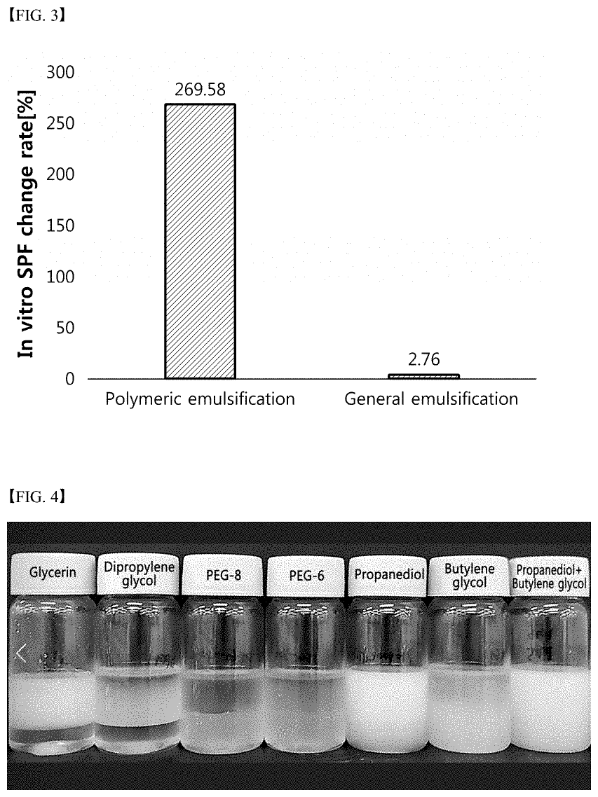

In order to evaluate the effect on the UV blocking effects of general emulsification; and polymeric emulsification using a polymer having both hydrophilic and hydrophobic groups in the chain and thus having both thickening ability and emulsifying ability at the same time, the compositions shown in Table 2 below were prepared and each UV blocking composition was applied to a PMMA plate, dried at a room temperature for 15 minutes and the initial in vitro SPF was measured. Then, it was heated in a 40° C. thermostat for 15 minutes and the subsequent in vitro SPF was measured, and the rate of change between the initial numerical value and the subsequent numerical value was calculated, and the result was shown in FIG. 3.

TABLE 2

| | | Polymeric | General |
|---|---|---|---|
| | Component name | emulsification | emulsification |
| Water phase | Water | To 100 | To 100 |
| | Butylene glycol | 6 | 6 |
| | Propanediol | 5 | 5 |
| | 1,2-hexanediol | 1.5 | 1.5 |
| | Polyacrylate-13 | 0.4 | — |
| | Hydroxyethylacrylate/sodium | 0.2 | — |
| | acryloyldimethyltaurate copolymer | | |
| | Ammonium acryloyldimethyltaurate/VP copolymer | 0.2 | — |
| | Polysorbate20(tween 20)/emulsifier | — | 1 |
| | Carbomer | — | 0.3 |
| Oil phase | Ethylhexylmethoxycinnamate | 6.8 | 6.8 |
| | Diethylaminohydroxybenzoylhexylbenzoate | 4 | 4 |
| | Bis-ethylhexyloxyphenolmethoxyphenyltriazine | 4 | 4 |
| | C12-15 alkylbenzoate | 8 | 8 |
| | Titanium dioxide | 2.45 | 2.45 |
| | Aluminum stearate | 0.42 | 0.42 |
| | Polyhydroxystearic acid | 0.35 | 0.35 |
| | Alumina | 0.14 | 0.14 |
| | Cyclopentasiloxane | 1.5 | 1.5 |
| | Silica | 1 | 1 |
| | Dimethicone | 2 | 2 |
| | Trisiloxane | 1.5 | 1.5 |

| | | |
|---|---|---|
| Total | 100 | 100 |
| In vitro SPF change rate (%) | 269.58 | 2.76 |

As a result of the experiment, it was confirmed that in case of the polymeric emulsification composition using a self-emulsifying polymer, compared to general emulsification, the in vitro SPF increase rate after heating was significantly high.

2. Confirmation of the Effect of Self-Emulsifying Polymer (SEP)

By using various self-emulsifying polymers having thickening ability and emulsifying ability at the same time by having hydrophilic and hydrophobic groups in the chain, the effect on the UV blocking effect was confirmed.

Specifically, experimental samples were applied to a PMMA plate (HelioScreen Labs, HD6) at 1.3 mg/cm², dried at room temperature for 15 minutes, and the initial in vitro SPF was measured by SPF-290AS (Solar light, USA). The in vitro SPF was measured in 6 different parts of the PMMA plate, and the average value was used. After that, the PMMA plate in which the initial in vitro SPF was measured was put on a hot plate already set at 40° C. and heated for 15 minutes, and then the subsequent in vitro SPF was measured, and the rate of change between the initial numerical value and subsequent numerical value was calculated.

In order to confirm the effect of the SEP polymers, the composition prepared by using a polymer having no emulsifying ability and an emulsifier, and the 14 kinds of compositions prepared by using a self-emulsifying polymer were examined for in vitro SPF value change rate by heat, and the result was shown in Table 3 and Table 4 below.

TABLE 3

| | | Comparative | | | | | | | |
|---|---|---|---|---|---|---|---|---|---|
| | Raw material name | example 1 | Example 1 | Example 2 | Example 3 | Example 4 | Example 5 | Example 6 | Example 7 |
| Water | Water | to 100 | to 100 | to 100 | to 100 | to 100 | to 100 | to 100 | to 100 |
| phase | Butylene glycol | 5 | 5 | 5 | 5 | 5 | 5 | 5 | 5 |
| | Propanediol | 5 | 5 | 5 | 5 | 5 | 5 | 5 | 5 |
| | 1,2-hexanediol | 1.5 | 1.5 | 1.5 | 1.5 | 1.5 | 1.5 | 1.5 | 1.5 |
| | Carbomer | 0.3 | | | | | | | |
| | Polysorbate 20 | 1 | | | | | | | |
| | Acrylate/C1O-30alkylacrylate | | 1 | | | | | | |
| | cross-polymer | | | | | | | | |
| | PEG-240/HDI copolymer bis- | | | 1 | | | | | |
| | decyltetradeceth-20ether | | | | | | | | |
| | Sodium polyacrylate | | | | 1 | | | | |
| | Ammonium acryloyldimethyltaurate/ | | | | | 1 | | | |
| | VP copolymer | | | | | | | | |
| | Ammonium acryloyldimethyltaurate/ | | | | | | 1 | | |
| | beheneth-25methacrylate cross-polymer | | | | | | | | |
| | Polyacrylate cross-polymer-6 | | | | | | | 1 | |
| | Hydroxyethylacrylate/sodium | | | | | | | | 1 |
| | acryloyldimethyltaurate copolymer | | | | | | | | |
| Oil | Ethylhexylmethoxycinnamate | 6.8 | 6.8 | 6.8 | 6.8 | 6.8 | 6.8 | 6.8 | 6.8 |
| phase | Diethylaminohydroxybenzoyl- | 4 | 4 | 4 | 4 | 4 | 4 | 4 | 4 |
| | hexylbenzoaate | | | | | | | | |
| | Bis-ethylhexyloxyphenolmethoxy- | 3 | 3 | 3 | 3 | 3 | 3 | 3 | 3 |
| | phenyltriazine | | | | | | | | |
| | C12-15 alkylbenzoate | 8 | 8 | 8 | 8 | 8 | 8 | 8 | 8 |
| | Titanium dioxide | 3 | 3 | 3 | 3 | 3 | 3 | 3 | 3 |
| | Cyclopentasiloxane | 2 | 2 | 2 | 2 | 2 | 2 | 2 | 2 |
| | Dimethicone | 3 | 3 | 3 | 3 | 3 | 3 | 3 | 3 |
| | Silica | 1 | 1 | 1 | 1 | 1 | 1 | 1 | 1 |

| | | | | | | | | |
|---|---|---|---|---|---|---|---|---|
| Total | 100 | 100 | 100 | 100 | 100 | 100 | 100 | 100 |
| in vitro SPF change rate (%) | −0.13 | 7.14 | 9.87 | 22.84 | 30.37 | 28.18 | 46.89 | 16.3 |

TABLE 4

| | Raw material name | Example 8 | Example 9 | Example 10 | Example 11 | Example 12 | Example 13 | Example 14 |
|---|---|---|---|---|---|---|---|---|
| Water | Water | to 100 | to 100 | to 100 | to 100 | to 100 | to 100 | to 100 |
| phase | Butylene glycol | 5 | 5 | 5 | 5 | 5 | 5 | 5 |
| | Propanediol | 5 | 5 | 5 | 5 | 5 | 5 | 5 |
| | 1,2-hexanediol | 1.5 | 1.5 | 1.5 | 1.5 | 1.5 | 1.5 | 1.5 |
| | Polyacrylate-13 | 1 | | | | | | |
| | Polyacrylamide | | 1 | | | | | |
| | Acrylate copolymer | | | 1 | | | | |
| | Acrylate/beheneth-25methacrylate | | | | 1 | | | |
| | copolymer | | | | | | | |
| | Sodium acrylate/acryloyldimetatrate/ | | | | | 1 | | |
| | dimethylacrylamide cross-polymer | | | | | | | |
| | Sodium acrylate/sodium | | | | | | 1 | |
| | acryloyldimethyltaurate copolymer | | | | | | | |
| | Sodium acrylate/beheneth- | | | | | | | 1 |
| | 25methacrylate cross-polymer | | | | | | | |
| Oil | Ethylhexylmethoxycinnamate | 6.8 | 6.8 | 6.8 | 6.8 | 6.8 | 6.8 | 6.8 |
| phase | Diethylaminohydroxybenzoyl- | 4 | 4 | 4 | 4 | 4 | 4 | 4 |
| | hexylbenzoate | | | | | | | |
| | Bis-ethylhexyloxyphenyltriazine | 3 | 3 | 3 | 3 | 3 | 3 | 3 |
| | C12-15 alkylbenzoate | 8 | 8 | 8 | 8 | 8 | 8 | 8 |
| | Titanium dioxide | 3 | 3 | 3 | 3 | 3 | 3 | 3 |
| | Cyclopentasiloxane | 2 | 2 | 2 | 2 | 2 | 2 | 2 |
| | Dimethicone | 3 | 3 | 3 | 3 | 3 | 3 | 3 |
| | Silica | 1 | 1 | 1 | 1 | 1 | 1 | 1 |

| | | | | | | | |
|---|---|---|---|---|---|---|---|
| Total | 100 | 100 | 100 | 100 | 100 | 100 | 100 |
| in vitro SPF change rate (%) | 10.49 | 11.23 | 15.32 | 8.68 | 16.7 | 20.23 | 17.68 |

As a result of the experiment, it was confirmed that in case of general emulsification (Comparative example 1), there was no change in the in vitro SPF numerical value before and after heating, but in case of the composition using the SEP, the in vitro SPF numerical value was increased by heat.

This was thought that in case of polymeric emulsification using the SEP, when the fluidity of the UV blocking agent was increased by a temperature increase, the movement of the UV blocking agent was not hindered, and the UV blocking area due to the movement of the UV blocking agent was expanded, thereby increasing the blocking effect.

3. Confirmation of the Effect of Polyol Having IOB Value of 5.0 or Less

In order to confirm the effect of the polyol, the in vitro SPF change rate before and after heating the composition was measured for a composition not comprising polyol in the water phase and for compositions comprising a polyol having IOB of 5.0 or less, where 6 polyols were selected from widely used polyols, and the result was shown in Table 5 below.

TABLE 5

| | | Comparative | | | |
|---|---|---|---|---|---|
| | Raw material name | example 2 | Example 15 | Example 16 | Example 17 |
| Water | Water | to 100 | to 100 | to 100 | to 100 |
| phase | Glycerin | | 10 | | |
| | Dipropylene glycol | | | 10 | |
| | PEG-6 | | | | 10 |
| | PEG-8 | | | | |
| | Propanediol | | | | |
| | Butylene glycol | | | | |
| | Polyacrylate-13 | 0.35 | 0.35 | 0.35 | 0.35 |
| | Hydroxyethylacrylate/sodium | 0.2 | 0.2 | 0.2 | 0.2 |
| | acryloyldimethyltaurate copolymer | | | | |
| | Ammonium acryloyldimethyltaurate/ | 0.2 | 0.2 | 0.2 | 0.2 |
| | VP copolymer | | | | |
| Oil | Ethylhexylmethoxycinnamate | 6.8 | 6.8 | 6.8 | 6.8 |
| phase | Diethylaminohydroxybenzoyl- | 4 | 4 | 4 | 4 |
| | hexylbenzoate | | | | |
| | Bis-ethylhexyloxyphenolmethoxy- | 3 | 3 | 3 | 3 |
| | phenyltriazine | | | | |
| | C12-15 alkylbenzoate | 8 | 8 | 8 | 8 |
| | Titanium dioxide | 3 | 3 | 3 | 3 |
| | Cyclopentasiloxane | 2 | 2 | 2 | 2 |
| | Dimethicone | 3 | 3 | 3 | 3 |
| | Silica | 1 | 1 | 1 | 1 |
| Total | | 100 | 100 | 100 | 100 |
| in vitro SPF change rate (%) | | −12.74 | 1.67 | 16.63 | 17.61 |
| | Raw material name | Example 18 | Example 19 | Example 20 | Example 21 |
| Water | Water | to 100 | to 100 | to 100 | to 100 |
| phase | Glycerin | | | | |
| | Dipropylene glycol | | | | |
| | PEG-6 | | | | |
| | PEG-8 | 10 | | | |
| | Propanediol | | 10 | | 5 |
| | Butylene glycol | | | 10 | 5 |
| | Polyacrylate-13 | 0.35 | 0.35 | 0.35 | 0.35 |
| | Hydroxyethylacrylate/sodium | 0.2 | 0.2 | 0.2 | 0.2 |
| | acryloyldimethyltaurate copolymer | | | | |
| | Ammonium acryloyldimethyltaurate/ | 0.2 | 0.2 | 0.2 | 0.2 |
| | VP copolymer | | | | |
| Oil | Ethylhexylmethoxycinnamate | 6.8 | 6.8 | 6.8 | 6.8 |
| phase | Diethylaminohydroxybenzoyl- | 4 | 4 | 4 | 4 |
| | hexylbenzoate | | | | |
| | Bis-ethylhexyloxyphenolmethoxy- | 3 | 3 | 3 | 3 |
| | phenyltriazine | | | | |
| | C12-15 alkylbenzoate | 8 | 8 | 8 | 8 |
| | Titanium dioxide | 3 | 3 | 3 | 3 |
| | Cyclopentasiloxane | 2 | 2 | 2 | 2 |
| | Dimethicone | 3 | 3 | 3 | 3 |
| | Silica | 1 | 1 | 1 | 1 |

| | | | | |
|---|---|---|---|---|
| Total | 100 | 100 | 100 | 100 |
| in vitro SPF change rate (%) | 34.49 | 62.55 | 137.3 | 269.58 |

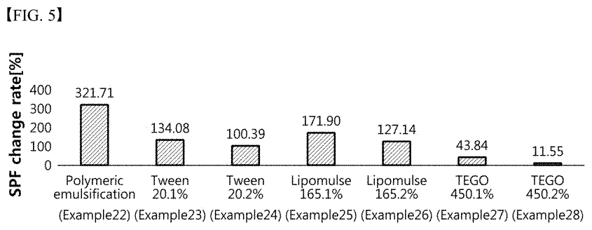

As a result of the experiment, it was confirmed that in case of the composition not comprising a polyol (Comparative example 2), the in vitro SPF numerical value was largely reduced when heat was applied, but in case of a composition comprising a polyol having an IOB value of 5.0 or less, the numerical value after heating was increased. In order to confirm the cause of the difference in the numerical value increase according to the polyol type, the polyols used in Examples 1521 and the oil phase excluding powder were mixed at a mass ratio of 1:1, and then the degree of separation was visually confirmed the next day, and the result of evaluating the compatibility on a 5-point scale was shown in Table 8 and FIG. 4 below.

TABLE 6

| | Glycerin | Dipropylene glycol | PEG-8 | PEG-6 | Propanediol | Butylene glycol | Propanediol + butylene glycol |
|---|---|---|---|---|---|---|---|
| Compatibility | 1 | 1.5 | 2 | 2 | 3 | 3 | 4 |

As a result of the experiment, it was confirmed that the better the compatibility between the oil phase and polyol, the higher the in vitro SPF change rate. Based on this, it could be confirmed that when the polyol having an IOB value of 5.0 or less was comprised, due to the compatibility between the UV blocking agent and the polyol remaining in the water phase after applying the formulation, expansion of the UV blocking agent into the water phase during heating was possible, and thus, an increase in the blocking effect occurred.

4. Confirmation of the Effect of Comprising an Emulsifier

In case of the polymeric emulsification composition using a self-emulsifying polymer, in order to compare the effect of addition of other emulsifiers, UV blocking compositions were prepared as Table 7, and the SPF change rate after heating was calculated. Specifically, each UV blocking composition was applied to a PMMA plate and dried for 15 minutes, and the initial in vitro SPF was measured. Then, after heating in a 40° C. thermostat for 15 minutes, the subsequent in vitro SPF was measured, and the SPF change rate compared to the initial numerical value was calculated, and the result was shown in Table 7 and FIG. 5.

TABLE 7

| | Component name | Example 22 | Example 23 | Example 24 | Example 25 | Example 26 | Example 27 | Example 28 |
|---|---|---|---|---|---|---|---|---|
| Water | Water | To 100 | To 100 | To 100 | To 100 | To 100 | To 100 | To 100 |
| phase | Butylene glycol | 6 | 6 | 6 | 6 | 6 | 6 | 6 |
| | Propanediol | 5 | 5 | 5 | 5 | 5 | 5 | 5 |
| | 1,2-hexanediol | 1.5 | 1.5 | 1.5 | 1.5 | 1.5 | 1.5 | 1.5 |
| | Polysorbate20 (Tween 20) | | 1 | 2 | | | | |
| | Glycerylstearate, PEG-100stearate | | | | 1 | 2 | | |
| | (Lipomulse 165) | | | | | | | |
| | Polyglyceryl- | | | | | | 1 | 2 |
| | 3methylglucosedistearate | | | | | | | |
| | (Tego care 450) | | | | | | | |
| | Polyacrylate-13 | 0.4 | 0.4 | 0.4 | 0.4 | 0.4 | 0.4 | 0.4 |
| | Hydroxyethylacrylate/sodium | 0.2 | 0.2 | 0.2 | 0.2 | 0.2 | 0.2 | 0.2 |
| | acryloyldimethyltaurate copolymer | | | | | | | |
| | Ammonium acryloyldimethyltaurate/ | 0.2 | 0.2 | 0.2 | 0.2 | 0.2 | 0.2 | 0.2 |
| | VP copolymer | | | | | | | |
| Oil | Ethylhexylmethoxycinnamate | 6.8 | 6.8 | 6.8 | 6.8 | 6.8 | 6.8 | 6.8 |
| phase | Diethylaminohydroxybenzoyl- | 4 | 4 | 4 | 4 | 4 | 4 | 4 |
| | hexylbenzoate | | | | | | | |
| | Bis-ethylhexyloxyphenol- | 4 | 4 | 4 | 4 | 4 | 4 | 4 |
| | methoxyphenyltriazine | | | | | | | |
| | C12-15 alkylbenzoate | 8 | 8 | 8 | 8 | 8 | 8 | 8 |
| | Titanium dioxide | 2.45 | 2.45 | 2.45 | 2.45 | 2.45 | 2.45 | 2.45 |
| | Aluminum stearate | 0.42 | 0.42 | 0.42 | 0.42 | 0.42 | 0.42 | 0.42 |
| | Polyhydroxystearic acid | 0.35 | 0.35 | 0.35 | 0.35 | 0.35 | 0.35 | 0.35 |
| | Alumina | 0.14 | 0.14 | 0.14 | 0.14 | 0.14 | 0.14 | 0.14 |
| | Cyclopentasiloxane | 1.5 | 1.5 | 1.5 | 1.5 | 1.5 | 1.5 | 1.5 |
| | Silica | 1 | 1 | 1 | 1 | 1 | 1 | 1 |
| | Dimethicone | 2 | 2 | 2 | 2 | 2 | 2 | 2 |
| | Trisiloxane | 1.5 | 1.5 | 1.5 | 1.5 | 1.5 | 1.5 | 1.5 |

| | | | | | | | |
|---|---|---|---|---|---|---|---|
| Total | 100 | 100 | 100 | 100 | 100 | 100 | 100 |
| In vitro SPF change rate (%) | 322 | 134 | 100 | 172 | 127 | 44 | 12 |

It was confirmed that in case of the polymeric emulsification compositions using a self-emulsifying polymer, the increase rate was reduced as the emulsifier was added, but in all, the in vitro SPF increase rate after heating was significantly higher than general emulsification Comparative example 1).

5. Confirmation of the Effect of Comprising an Oil Phase Thickener

The self-emulsifying polymer of the present disclosure is an aqueous thickener, and is comprised in the water phase. Accordingly, UV blocking compositions were to be prepared using an oil phase thickener instead of the self-emulsifying polymer of the present disclosure, but it was confirmed that in this case, the stability of the formulation was largely reduced. Thus, the effect of the self-emulsifying polymer of the present disclosure was compared with that of the case of comprising an oil phase thickener in addition to the aqueous thickener, and the result was shown in Table 8

TABLE 8

| | Component name | Composition 1 | Composition 2 | Composition 3 | Composition 4 | Composition 5 | Composition 6 |
|---|---|---|---|---|---|---|---|
| Water | Water | To 100 | To 100 | To 100 | To 100 | To 100 | To 100 |
| phase | Propanediol | 5 | 5 | 5 | 5 | 5 | 5 |
| | Butylene glycol | 5 | 5 | 5 | 5 | 5 | 5 |
| | Hydroxyethylacrylate/sodium | 0.35 | 0.35 | 0.35 | 0.35 | 0.35 | 0.35 |
| | acryloyldimethyltaurate copolymer | | | | | | |
| | Polyacrylate cross-polymer-6 | 0.2 | 0.2 | 0.2 | 0.2 | 0.2 | 0.2 |
| Oil | Ethylhexylmethoxycinnamate | 6.8 | 6.8 | 6.8 | 6.8 | 6.8 | 6.8 |
| phase | Diethylaminohydroxybenzoyl- | 4 | 4 | 4 | 4 | 4 | 4 |
| | hexylbenzoate | | | | | | |
| | Bis-ethylhexyloxyphenol- | 3 | 3 | 3 | 3 | 3 | 3 |
| | methoxyphenyltriazine | | | | | | |
| | C12-15 alkylbenzoate | 8 | 8 | 8 | 8 | 8 | 8 |
| | Dextrin palmitate | | 1 | | | | |
| | Disteardimoniumhectorite | | | 1 | | | |
| | Stearoyl inulin | | | | 1 | | |
| | Polyamide-8 | | | | | 1 | |
| | Glycerylbehenate/eicosadioate | | | | | | 1 |
| | Titanium dioxide | 3 | 3 | 3 | 3 | 3 | 3 |
| | Cyclopentasiloxane | 2 | 2 | 2 | 2 | 2 | 2 |
| | Dimethicone | 3 | 3 | 3 | 3 | 3 | 3 |
| | Silica | 1 | 1 | 1 | 1 | 1 | 1 |

| | | | | | | |
|---|---|---|---|---|---|---|
| Total | 100 | 100 | 100 | 100 | 100 | 100 |
| In vitro SPF change rate (%) | 82.20 | 32.78 | 20.89 | 48.16 | 14.66 | 40.21 |

As a result of the experiment, it was confirmed that the SPF increase rate was significantly reduced when various oil phase thickeners (Compositions 2 to 6) were added to composition 1, which was prepared only with the self-emulsifying polymer

6. Confirmation of the Effect of UV Irradiation

The result of confirming the in vitro SPF change according to the temperature when exposed to UV for the UV blocking composition of the present disclosure (Composition 1) was shown in Table 9

Specifically, experimental samples were applied to a PMMA plate (HelioScreen Labs, HD6) at 1.3 mg/cm², dried at a room temperature for 15 minutes, and the initial in vitro SPF was measured by SPF-290AS (Solar light, USA). After that, the PMMA plate in which the initial in vitro SPF was measured was put on a hot plate already set at 40° C. and heated for 15 minutes, and the subsequent in vitro SPF was measured, and the rate of change between the initial numerical value and the subsequent numerical value was caluated. Then, the in vitro SPF change rate according to the presence or absence of 2MED UV irradiation was confirmed using a UV irradiator (16S-300, Solar light).

TABLE 9

| In vitro SPF | Temperature | | |
|---|---|---|---|
| change rate | 25° C. | 30° C. | 40° C. |
| No UV irradiation | 2.13 | 18.65 | 82.20 |
| 2MED irradiation | 5.00 | 25.2 | 115.71 |

As a result of the experiment, it was confirmed that the in vitro SPF change rate was increased as the temperature increased, and the synergistic effect was more remarkable during UV irradiation.

1-11. (canceled)

12. A method of increasing UV light blocking efficiency of a UV light blocking agent under heat, comprising: adding a combination of (a) a self-emulsifying polymer and (b) a polyol having an IOB value of 5.0 or less to a UV light blocking agent to obtain a UV blocking composition as active ingredients for increasing UV light blocking efficiency of the UV blocking agent; applying the UV blocking composition to the skin of a subject, and exposing the skin of the subject under heat.

13. The method of claim 12, wherein the method comprises: preparing a water phase comprising the self-emulsifying polymer and the polyol; preparing an oil phase comprising the UV blocking agent; obtaining a UV blocking composition by mixing the water phase and the oil phase; and placing the composition under heat.

14. A UV light blocking composition for increasing UV light blocking efficiency of a UV light blocking agent under heat, comprising a combination of (a) a self-emulsifying polymer and (b) a polyol having an IOB value of 5.0 or less, as active ingredients for increasing the UV light blocking efficiency, and a UV light blocking agent.

15. The composition of claim 14, wherein the self-emulsifying polymer is selected from the group consisting of sodium acrylate/beheneth-25 methacrylate cross-polymer, sodium acrylate/sodium acryloyldimethyltaurate copolymer, sodium acylate/acryloyldimetatrate/dimethylacrylamidecross-polymer, sodium polyacrylate, acrylate/C10-30 alkylacrylate cross-polymer, acrylate/beheneth-25 methacrylate copolymer, acrylate copolymer, ammonium acryloyldimethyltaurate/VP copolymer, polyacrylate-13, polyacrylate cross-polymer-6, polyacrylamide, PEG-240/HDI copolymer bis-decyltetradeceth-20 ether, hydroxyethylacrylate/sodium acryloyldimethyltaurate copolymer, and combinations thereof.

16. The composition of claim 14, wherein the polyol having an IOB value of 5.0 or less is selected from the group consisting of glycerin, sorbitol, xylitol, glucose, trehalose, diglycerin, propandiol, propylene glycol, polyglycerin-3, methylpropandiol, butylene glycol, pentylene glycol, PEG-6, PEG-8, glycereth-26, dipropylene glycol, 1,2-hexanediol, caprylyl glycol, and combinations thereof.

17. The composition of claim 14, wherein the UV blocking agent is selected from the group consisting of aminobenzoic acid-based compounds, benzophenone-based compounds, cinnamate-based compounds, salicylate-based compounds, inorganic metal oxides-based compounds, butyl methoxydibenzoylmethane-based compounds, terephthalylidene dicamphor sulfonic acid ecamsule, phenylbenzimidazole sulfonic acid, bemotrizinol, bisoctrizole, and combinations thereof.

18. The composition of claim 14, wherein the self-emulsifying polymer is an aqueous thickener.

19. The composition of claim 14, wherein the composition comprises a water phase and an oil phase, and the water phase includes the combination of (a) self-emulsifying polymer and (b) polyol having an IOB value of 5.0 or less, and the oil phase includes the UV blocking agent.

20. The composition of claim 14, wherein the composition has synergistically increased UV blocking efficiency of the UV blocking agent under heat where UV rays are irradiated.
